(19) United States
(12) Patent Application Publication
(10) Pub. No.: US 2021/0275609 A1
(43) Pub. Date: Sep. 9, 2021

(54) PREVENTING FUNGAL GROWTH IN HONEY BEE COLONIES AND RELATED MATERIALS AND METHODS

(71) Applicants: Irene Lucile Garcia NEWTON, Bloomington, IN (US); Delaney L. MILLER, Bloomington, IN (US); Audrey J. PARISH, Bloomington, IN (US); The Trustees of Indiana University, Bloomington, IN (US)

(72) Inventors: Irene Lucile Garcia Newton, Bloomington, IN (US); Delaney L. Miller, Bloomington, IN (US); Audrey J. Parish, Bloomington, IN (US)

(73) Assignee: The Trustees of Indiana University, Bloomington, IN (US)

(21) Appl. No.: 17/259,457

(22) PCT Filed: Jul. 11, 2019
(86) PCT No.: PCT/US2019/041454

§ 371 Date: Jan. 11, 2021

Related U.S. Application Data
(60) Provisional application No. 62/697,232, filed on Jul. 12, 2018.

(51) Int. Cl. A61K 35/741; A61P 31/10

(52) U.S. Cl. CPC A61K 35/741; A61P 31/10

(57) ABSTRACT

Compositions and methods for inhibiting fungal pathogen growth in honey bee colonies are provided. In particular, compositions include at least one strain of Parasaccharibacter apium (P. apium), a supernatant of the one or more strains of P. apium, or a combination of the one or more strains of P. apium and the supernatant of the one or more strains of P. apium. One or all of the strains of P. apium can be resistant to one or more antibiotics (e.g., kanamycin), and may include an exogenous antibiotic resistance-conferring transposon, gene, or functional fragment thereof. Also provided are methods, the methods generally including the step of applying an effective amount of a composition to a honey bee colony. Compositions can be fed to the bees, deposited on the hive frame, deposited on at least one surface or element within the hive, or a combination thereof.

### BACKGROUND

The European honey bee (Apis melliferia) is heavily utilized in agriculture for both pollination efforts and for the production of honey. As pollinators of many major crops, honey bees add billions of dollars annually to the US economy. The decline in population size of this agricultural insect has increased both interest and research into honey bees. The alarming and dramatic decline of the honey bee is likely due to the influence of important environmental stressors on honey bee health, productivity, and longevity. These stressors include limited floral resources (nutritional stress), parasites and pathogens (immune stress), and exposure to fungicides and pesticides (chemical stress). Additionally, it is becoming clear that the combination of stressors, simultaneously experienced by honey bees, may act synergistically to further push honey bees past the tipping point, increasing colony decline.

One factor that may influence honey bee and honey bee colony health is the microbial community populating the honey bee and honey bee colonies.

Honey bee colonies are vulnerable to pathogens, including fungi. Notably, fungal pathogens can render weakened colonies further susceptible to infestations by other pests and/or pathogens. The cumulative effect can result in the destruction of the hive.

Declines in overall honey bee health (and thus populations) and increased drug resistance by evolving pathogens has created a demand for new approaches capable of directly inhibiting fungal growth in honey bee colonies.

### SUMMARY

In a first example (“Example 1”), described herein is a composition comprising one or more strains of Parasaccharibacter opium (P. apium) a supernatant of the one or more strains of P. apium, or a combination of the one or more strains of P. apium and the supernatant of the one or more strains of P. apium.

In another example (“Example 2”) further to Example 1, the one or more strains of P. apium comprises strain SME1.

In another example (“Example 3”) further to Example 1 or 2, the one or more strains of P. apium comprises one or more antibiotic resistant strains.

In another example (“Example 4”) further to any one of Examples 1-3, at least one of the one or more antibiotic resistant strains is resistant to kanamycin.

In another example (“Example 5”) further to any one of Examples 1-4, at least one of the one or more antibiotic resistant strains comprises an exogenous antibiotic resistance transposon, gene, or a functional fragment thereof.

In another example (“Example 6”) further to any one of Examples 1-5, at least one of the one or more antibiotic resistant strains comprises an exogenous kanamycin resistance transposon, gene or a functional fragment thereof.

In another example (“Example 7”) further to Example 5 or 6, the exogenous kanamycin resistance gene or functional fragment thereof comprises a mini-Tn5 KanR cassette

In another example (“Example, 8”) further to Example 7, the at least one of the one or more antibiotic resistant strains comprising the mini-Tri5 Kara cassette is an SME1 mutant.

In another example (“Example 9”) further to any one of Examples 1-8, the composition is aqueous.

In another example (“Example 10”) further to any one of Examples 1-9, the composition comprises a liquid carrier or a gel-based carrier.

In another example (“Example 11”) further to any one of Examples 1-10, the composition comprises at least one carbon source.

In a second example (“Example 12”), described herein is a method for inhibiting fungal growth in a honey bee colony, the method comprising applying an effective amount of a composition of any one of one of claims 1-11.

In another example (Example 13), further to Example 12, wherein applying the effective amount of the composition to a honey bee colony comprises at least one of: feeding the composition to honey bees of the honey bee colony; depositing the composition on a frame of the honey bee colony's hive, and depositing the composition on at least one surface or element within the honey bee colony's hive.

### BRIEF DESCRIPTION OF THE DRAWINGS

FIG. 1 is a collection of photographs demonstrating in vitro competition between acetic acid bacteria (AAB) and pathogenic fungus Beauverio bassiano (B. bassiana) according to one embodiment.

FIG. 2 is a cartoon illustrating an antifungal assay according to one embodiment.

FIG. 3 is a line graph representing the results from the antifungal assay of FIG. 2.

FIG. 4 is a group of two line graphs representing the results from experiments involving compounds secreted by P. apium according to one embodiment.

FIG. 5 is a cartoon illustrating an antifungal assay according to one embodiments.

FIGS. 6A and 6B are line graphs representing the results from the antifungal assay of FIG. 5.

FIG. 7 is a cartoon illustrating an antifungal assay according to one embodiment.

FIG. 8 is a line graph representing the results from the antifungal assay of FIG, 7.

FIG. 9 is a cartoon illustrating a fungal pathogen challenge assay according to one embodiment.

FIG. 10 presents a survival time course for honey bees challenged with a pathogenic fungus according to one embodiment.

FIG. 11 is a box plot indicating spore number in bee pupae infected with P. apium under different conditions according to one embodiment.

FIG. 12 is a box plot indicating the ability of honey bee microbiome to protect bee larvae from nutritive stress according to one embodiment.

### DETAILED DESCRIPTION

Certain embodiments described herein provide compositions capable of inhibiting growth of fungal pathogens in honey bee hives, as well as improve survival in honey bees subjected to a fungal pathogen. Compositions of the present disclosure generally comprise one or more strains of Parasaccharibacter apium (P. apium), a supernatant of the one or more strains of P. apium, or a combination of the one or more strains of P. apium and the supernatant of the one or more strains of P. apium.

P. apium is part of a Glade of acetic acid bacteria (AAB—a group within the family Acetobacteraceae) that contains both free-living and bee-associated members, and is one of the primary bacteria that differentiate the microbial community associated with queen bees compared to that associated with worker bees. The bacterium occupies defined niches within the hive, including queen guts, nurse hypopharyngeal glands, nurse crops, and royal jelly. As described herein, P. apium inhibits growth of fungal pathogens in honey bee hives and improves survival in honey bees subjected to fungal pathogens.

In certain embodiments, the compositions inhibit growth of honey bee fungal pathogens such as, for example, Nosema apis, Ascophera apis (chalkbrood), Aspergillus flavus (stonebrood), and Beauveria bassiana. Additional honey bee fungal pathogens may similarly be inhibited by compositions described herein.

In some embodiments, strains of P. apium are isolated from a honey bee colony. P. apium can be isolated from, for example, the nectar, beebread, larval diet, and queen bees, as the bacterium is consistently present in these specific environments. In certain embodiments, a composition includes a single P. apium strain, in other embodiments, the composition includes multiple P. apium strains.

In some embodiments, a supernatant of a P. apium strain(s) is included in the composition. The supernatant can be, for example, the spent media following culture of the P. apium strains or a supernatant collected following lysis of the P. apium. Spent media following culture of P. apium can easily be collected during cell culture. The supernatant to be collected following lysis of the P. apium can be produced and collected by, for example, disrupting the membranes of the bacteria (e.g., by sonication), separating the aqueous phase from the solid phase (e.g., by centrifugation), and collecting the aqueous phase as the supernatant. In some embodiments, the supernatant is treated with a protease. In certain embodiments, protease treatment does not affect the fungal growth inhibitory properties of the supernatant, and eliminates unwanted proteins, including enzymes, from the supernatant.

In other embodiments, the composition includes both the P. apium strains) and the supernatant of P. apium.

It will be recognized that various combinations of P. apium strains and their supernatants can be included in the compositions. For example, a composition may include a first P. apium strain and the supernatant of a second P. apium strain, include both the first and second P. apium strains and the supernatant of neither strain, include only the supernatant of both the first and second P. apium strains, include both the first and second P. apium strains and the supernatant of either the first or second P. apium strain, include both the first and second P. apium strains and the supernatant of both strains. By increasing the number of strains and/or supernatants thereof, the possible combinations are many.

In certain embodiments, the composition includes P. apium strain SME1, a deposit of which is maintained by Dr. Irene Newton, Department of Biology, Indiana University, Bloomington, 107 S. Indiana Ave, Bloomington, Ind. 47405, which will be deposited in accordance with the Budapest Treaty and with U.S. 37 C.F.R. §§ 1.801-1.809. The accession number will be provided upon receipt thereof. Following deposit with the ATCC, access to this deposit will be available during the pendency of this application to persons determined by the Commissioner of Patents and Trademarks to be entitled thereto under 37 CFR § 1.14 and 35 USC § 122.

Certain P. apium strains demonstrate increased fungal inhibitory capacity, while others are better suited to culture in the lab. P. apium was identified as being capable of inhibiting fungal growth while also being readily cultured in the laboratory.

In some embodiments, the P. apium strain(s) to be included in the composition is resistant to antibiotics, including, for example, kanamycin, tetracycline, oxytetracyline, tylosin, and lincomycin, although resistance to other antibiotics used in apiculture or found to contaminate honey bee colonies may also be desirable. In certain embodiments, the P. apium strain(s) to be included in the composition is resistant to kanamycin.

Antibiotic resistance may be selected for or intentionally conferred to the P apium strain(s). in some embodiments, an antibiotic resistant mutant may be identified and selected for by growing a strain of P. apium on a selective media including the antibiotic to which resistance is desired. Those colonies capable of growing on the selective media are then selected for further development and testing to ensure established antibiotic resistance. In other embodiments, antibiotic resistance is intentionally introduced to a bacterial strain by incorporating an exogenous antibiotic resistance-encoding transposon, gene, or a fractional fragment thereof (i.e., a fragment of the transposon or the gene) into the bacteria.

Several antibiotic resistance-encoding transposons and genes are known in the art. For example, transposon Tn5 encodes resistance to kanamycin, while transposon Tn10 encodes resistance to tetracycline. Methods useful for integrating the antibiotic resistance-encoding exogenous cassette include transposition and transformation. In some embodiments, a mini-Tn5 KanR (kanamycin resistance) cassette is introduced to P. apium via transposition. Transposition call utilize, for example, a mating strain of E. coil harboring a Tn5 transposon on a suicide plasmid backbone (e.g., pWN93), ensuring that those selected colonies have integrated the transposon. In particular embodiments, the P. apium strain is an SME1 transposon mutant that incorporates a mini-Tn5 KanR cassette. A representative example of such an SME1 mutant is maintained by Dr. Irene Newton, Department of Biology, Indiana University, Bloomington, 107 S. Indiana Ave, Bloomington, Ind. 47405, which will be deposited in accordance with the Budapest Treaty and with U.S. 37 C.F.R. §§ 1.801-1.809. The accession number will be provided upon receipt thereof. Following deposit with the ATTC, access to this deposit will be available during the pendency of this application to persons determined by the Commissioner of Patents and Trademarks to be entitled thereto under 37 CFR § 1.14 and 35 USC § 1122.

In some embodiments, compositions including one or more strains of P. apium, a supernatant of the one or more strains of P. apium, or a combination of the one or more strains of P. apium and the supernatant of the one or more strains of P. apium (i.e., the active element) are aqueous. In some embodiments, the composition includes an acceptable carrier. An acceptable carrier should be non-toxic to the active element (i.e., the P. apium) and to honey bees, and may promote viability to the active element during storage. The carrier can be a liquid carrier or a gel-based carrier. Examples of appropriate carriers include, but are not limited to water, physiological electrolyte solutions, and glycols such as methanol, ethanol, propanol, butanol, ethylene glycol, and propylene glycol. In some embodiments, the composition includes only water and the active element. Compositions may be, for example, in the form of a liquid suspension, a paste, a syrup, or a gel. Compositions will generally be homogenous.

In certain embodiments, the composition also includes one or more carbon sources, which may act as a nutrient source for honey bees, P. apium included in the composition, or both, Non-limiting examples of carbon sources include fructose, glucose, sucrose, maltose, galactose, sorbitol, xylan, pectin, and lignin.

Also provided are methods for inhibiting fungal growth in a honey bee colony and/or hive, and/or improving survival in honey bees challenged with a fungal pathogen. Such methods generally include applying an effective amount of a composition described herein to a honey bee colony.

In some embodiments, applying the effective amount of the composition to a honey bee colony comprises feeding the composition to honey bees of the honey bee colony, wherein an effective amount of the composition is placed in or near a honey bee colony's hive so that the honey bees can feed on the composition. Methods for feeding honey bees include, for example, use of a frame feeder, and using a simple shallow tray, a bag feeder, or a jar feeder filled with the composition. In other embodiments, the composition can be deposited on the frame of the honey be colony's hive. In yet other embodiments, the composition can be deposited on at least one surface or element within the honey bee colony's hive, such as, for example, the nectar, beebread, brood cell, and larval feed. Depending on the consistency of the composition (e.g., liquid vs gel), the composition can be deposited by, for example, spraying, dripping, spreading, and coating.

As used herein, “effective amount” refers to an amount of a composition of the present disclosure that is sufficient to achieve a desired result (e.g., inhibit fungal growth, improve survival of bees) in a honey bee colony and/or its hive. An effective amount of the composition can be provided or applied in a single application, or in multiple applications. The effective amount may vary and depend on such factors such as colony size (i.e., population), method of applying the composition, and the desired result. The effective amount can be determined or modified by those of skill in the art.

### EXAMPLES

The materials, methods, and embodiments described herein are further defined in the following Examples. Certain embodiments are defined in the Examples herein. It should be understood that these Examples, while indicating certain embodiments, are given by way of illustration only. From the disclosure herein and these Examples, one skilled in the art can ascertain the essential characteristics of this invention, and without departing from the spirit and scope thereof, can make various changes and modifications of the invention to adapt it to various usages and conditions.

### EXAMPLE 1

In one example of the embodiments described herein, it is demonstrated that P. apium and supernatants thereof are capable of inhibiting fungal growth in vitro.

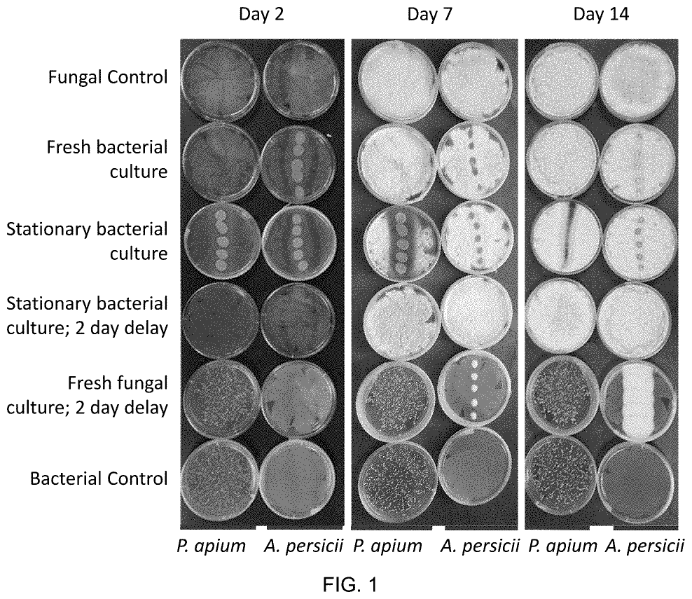

Turning to FIG. 1, competition between acetic acid bacteria (AAB; P. apium or Acetobacter persicii) and honey bee fungal pathogen Beauveria bassiana (B. bassiana) was examined in vitro. P. apium demonstrated much stronger fungal inhibition than A. persicii. As pictured in FIG. 1, B. bassiana was unable to establish on plates colonized with P. apium. Inhibition of B. bassiana was observed to be dose-dependent.

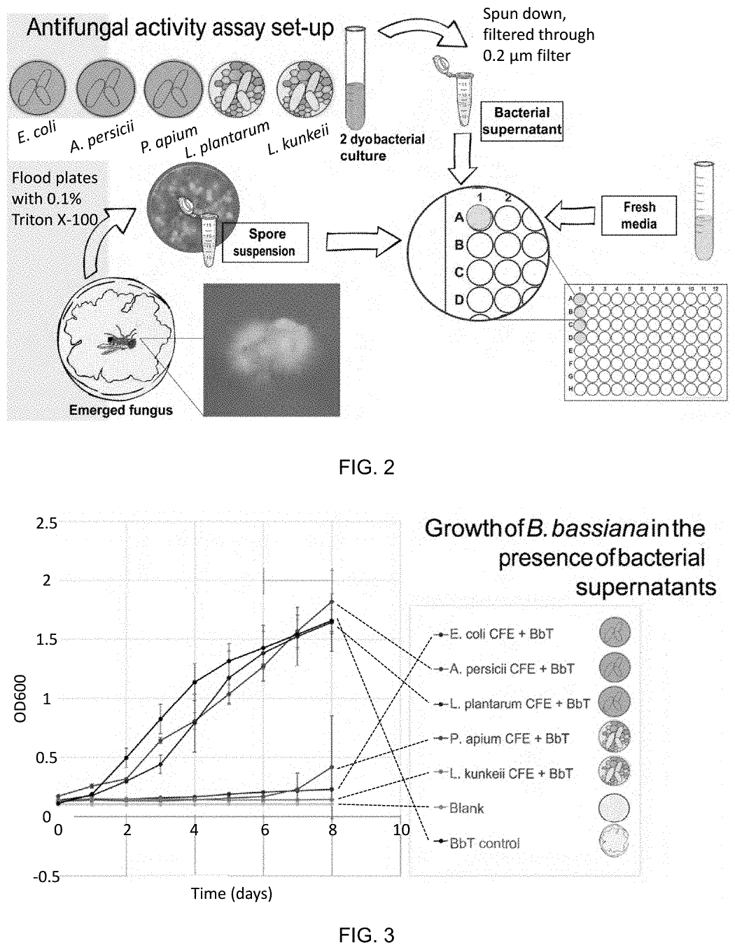

FIG. 2 illustrates a design for an antifungal assay. Fungus was allowed to emerge and colonize a culture plate from an infected fly (Drosophila melanogaster). Plates were then flooded and spores collected in a spore suspension. Supernatants of the noted bacterial were then combined with the spore suspension and fresh media in 96-well plates for incubation and analysis.

FIG. 3 presents a summary of the data collected from the inhibition experiment of FIG. 2. After 8 days, supernatant (i.e., cell free extract, or CFE) of A. persicii and L. plantarum did not inhibit fungal growth, while supernatant of P. apium, L. kunkeii, and E. coli each significantly inhibited fungal growth. The anti-fungal properties of E. coil and L. kunkeii against certain bacteria are known in the art. These data demonstrate the ability of P. apium to inhibit the fungal pathogen B. bassiana.

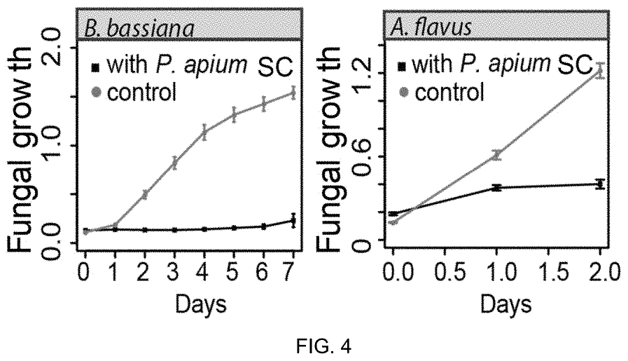

FIG. 4 presents a summary of the data collected in experiments where either B. bassiana or A. flavus was incubated in liquid culture in the presence of secreted compound (“SC”; i.e., spent media collected following culture) from P. apium. Growth of the fugus was measured over time. The secreted compound of P. apium demonstrated strong inhibitory activity against the growth of both B. bassiana and A. flavus.

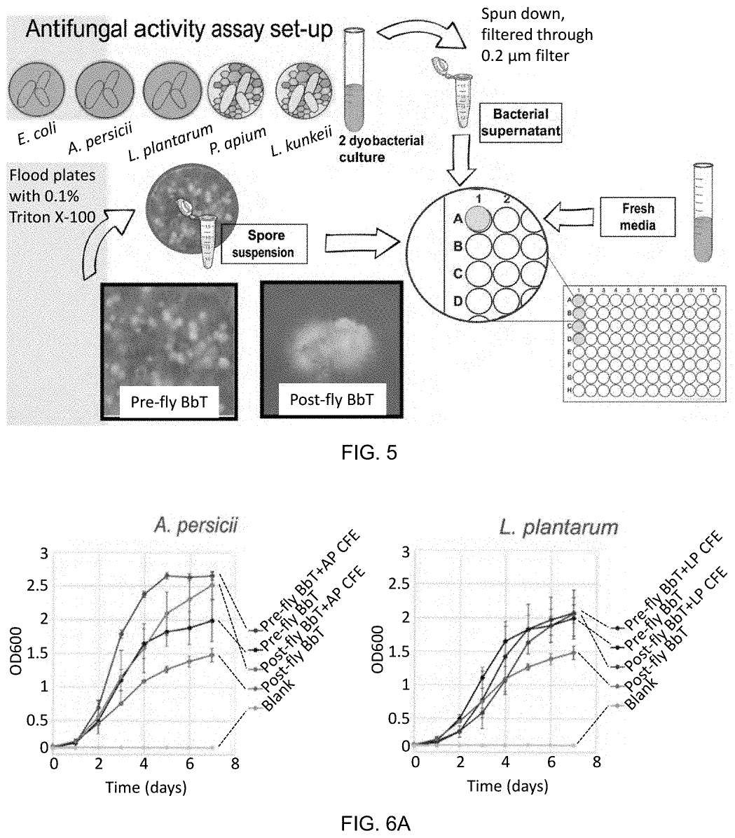

FIG. 5 illustrates a design for a second antifungal assay, wherein the effect of the noted bacterial supernatant on either pre-fly B. bassiana (BbT) (i.e., isolated fungus) or post-fly B. bassiana (i.e., fungus emerging from an infected fly) was examined.

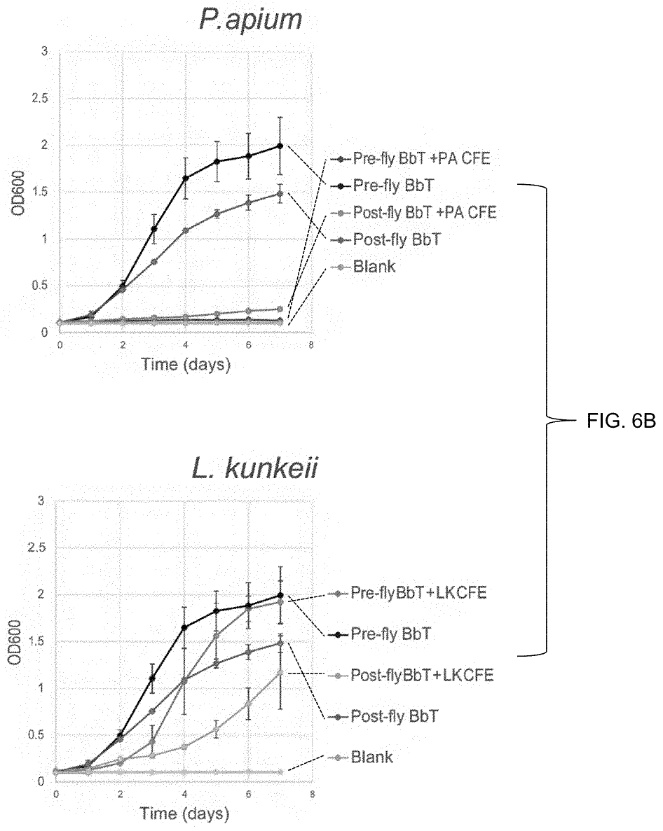

FIG. 6A presents a summary of the data collected from the inhibition experiment of FIG. 5 collected using the supernatant of D. melanogaster isolates A. persicii or L. plantarum. As depicted, supernatant of neither A. persicii or L. plantarum effectively inhibited B. bassiana. Interestingly, post-fly B. bassiana did not grow as well as pre-fly fungus in the absence of the bacterial isolate, suggesting that passaging the fungus through the fly affects pathogen virulence. A. persicii tended to improve growth of both pre- and post-fly fungus, while L. plantarum had no effect on pre-fly fungus, and may have slightly enhanced growth of post-fly fungus.

FIG. 6B presents a summary of the data collected from the inhibition experiment of FIG. 5 collected using the supernatant of A. mellifera isolates P. apium and L. kunkeii. As depicted, P. apium supernatant significantly inhibited the growth of both pre-and post-fly B. bassiana. After 7 days, supernatant of L. kunkeii had little effect on the total growth of either pre- or post-fly B. bassiana after 7 days, although the bacterial supernatant appears to have slowed the fungus' growth for the first few days.

These data demonstrate that the supernatant of P. apium is capable in and of itself to inhibit the growth of honey bee fungal pathogens.

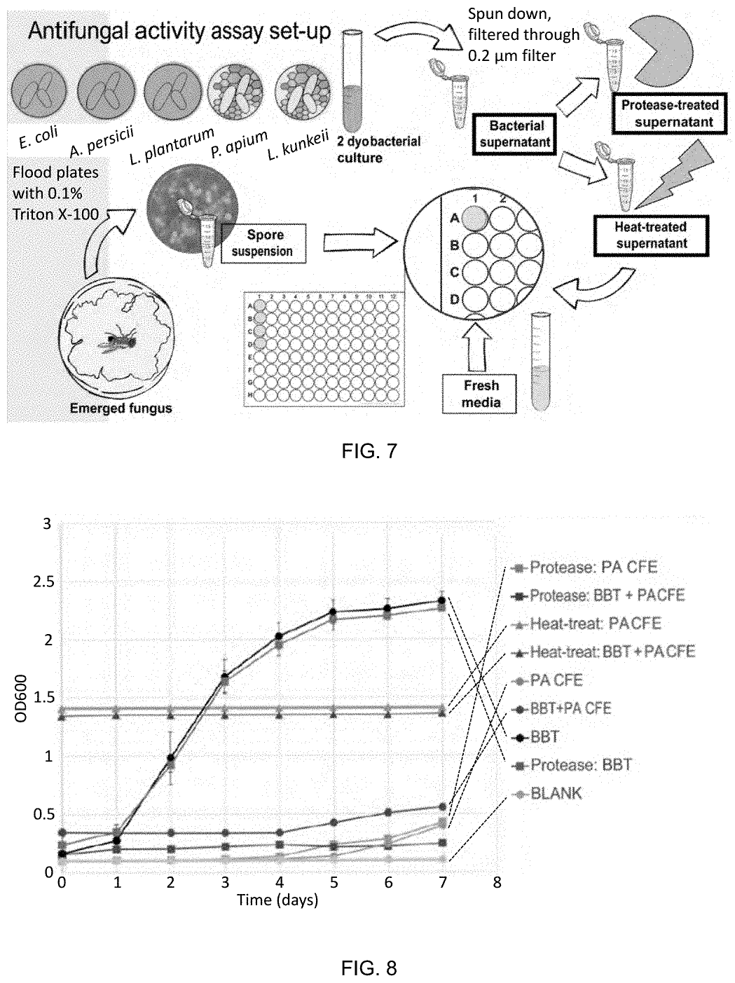

FIG. 7 illustrates a design for a third antifungal assay, wherein the effect of protease or heat treatment on the fungal growth inhibitory effect of bacterial supernatants. Bacterial supernatants were subjected to either protease or heat treatment, and combined with post-fly B. bassiana and fresh media in a 96-well plate for incubation and analysis.

FIG. 8 presents a summary of the data collected from the inhibition experiment of FIG. 7. As depicted, protease treatment had no direct effect on B. bassiana, and did not affect the ability of P. apium supernatant to inhibit fungal growth. These data suggest that the secreted compound from P. apium having fungal growth inhibitory properties is not a protein.

### EXAMPLE 2

In a second example of the embodiments described herein, it is demonstrated that P. apium and supernatants thereof are capable of improving pupal survival when challenged with pathogenic fungus and reduce pathogenic fungal spore load in infected pupae.

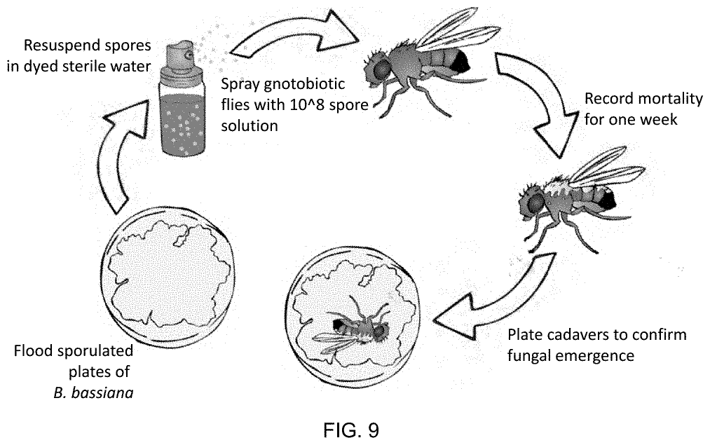

FIG. 9 illustrates a general design for an experiment to determine the susceptibility of an insect (e.g., D. melanogaster or A. melliferia) to pathogenic fungus. As depicted, spores of B. bassiana are collected and resuspended in water. Flies are then sprayed with the spore solution, and mortality is recorded for one week. Cadavers are plated to confirm fungal emergence. P. apium or a supernatant thereof can be applied to or fed to the insects to determine whether the bacterium or its supernatant has any inhibitory effect in adult insects.

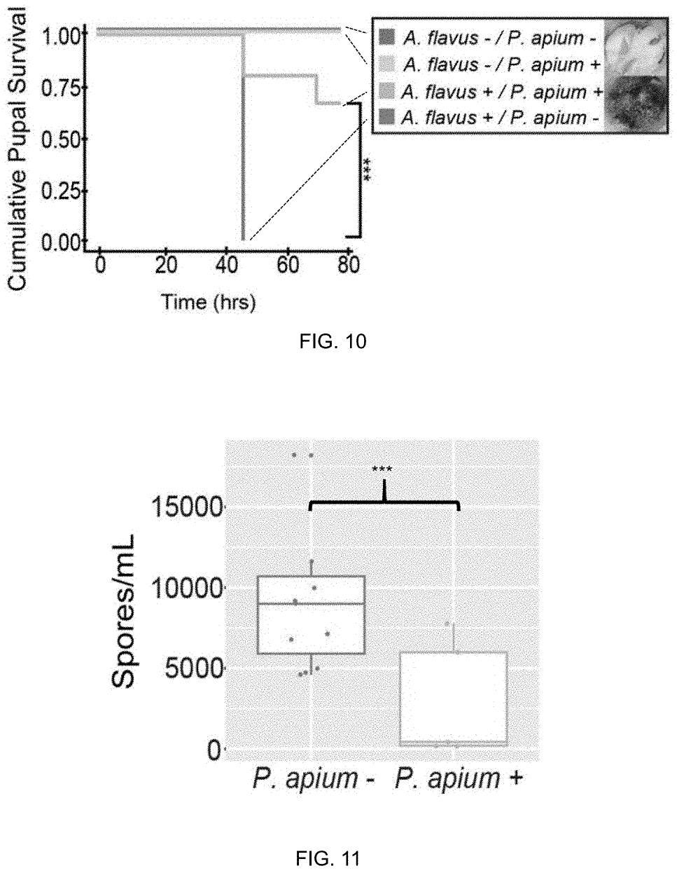

FIG. 10 presents the results from an experiment in which larvae collected as first-instars were fed larval diet with or without P. apium for three days prior to infection with A. flavus. Pupae were inoculated with spores of A. flavus (10⁵ spores/mL). Survival in P. apium-treated groups was significantly higher than in the untreated groups.

FIG. 11 presents the results from an experiment in which A. flavus spore numbers were quantified in infected bee pupae that were either treated with (i.e., fed) P. apium or not. As depicted, presence of P. apium significantly reduces A. flavus spore load during infection.

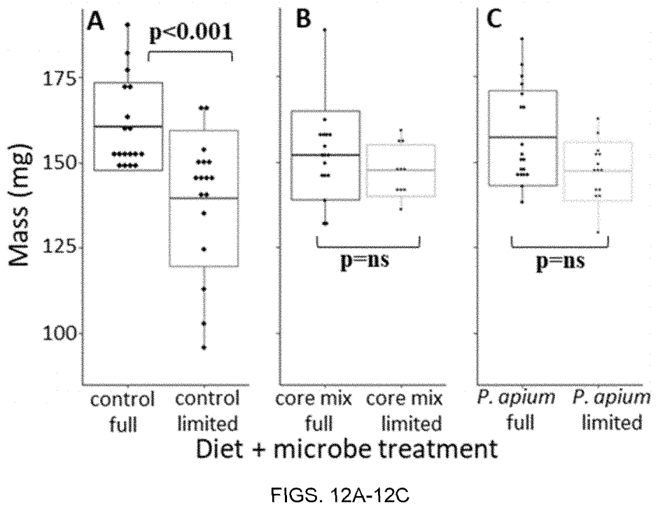

FIG. 12 presents the results from an experiment designed to investigate the effect of P. apium during nutrient stress in honey bees. To determine the effect of microbial symbionts on larval nutrition, honey bee larvae were raised on sterile diet containing either limited or full nutrition and supplemented a subset with a mix of four core microbiome members (F. fructosis, L. kunkeei, Bifidobacterium sp., and P. apium). The influence of P. apium alone on larval growth under these nutritionally stressed conditions was also tested. As depicted, larvae raised on limited nutrition without microbes were significantly smaller than those an full nutrition (FIG. 12A). However, if larvae were supplemented with core microbiome members, low-nutrition larvae were just as large as high-nutrition larvae (FIG. 12B). This effect was recapitulated in the larvae given P. apium alone (FIG. 12C). These results suggest that the microbiome may protect honey bee larvae from nutritive stress, that P. apium alone could be a crucial component of that protection, and that the beneficial effects of P. apium extend beyond its antifungal properties.

While the disclosed subject matter is amenable to various modifications and alternative forms, specific embodiments are described herein in detail. The intention, however, is not to limit the disclosure to the particular embodiments described. On the contrary, the disclosure is intended to cover all modifications, equivalents, and alternatives falling within the scope of the disclosure as defined by the appended claims.

Similarly, although illustrative methods may be described herein, the description of the methods should not be interpreted as implying any requirement of, or particular order among or between, the various steps disclosed herein. However, certain embodiments may require certain steps and/or certain orders between certain steps, as may be explicitly described herein and/or as may be understood from the nature of the steps themselves (e.g., the performance of some steps may depend on the outcome of a previous step). Additionally, a “set,” “subset,” or “group” of items (e.g., inputs, algorithms, data values, etc.) may include one or more items, and, similarly, a subset or subgroup of items may include one or more items. A “plurality” means more than one.

As the terms are used herein with respect to ranges, “about” and “approximately” may be used, interchangeably, to refer to a measurement that includes the stated measurement and that also includes any measurements that are reasonably close to the stated measurement, but that may differ by a reasonably small amount such as will be understood, and readily ascertained, by individuals having ordinary skill in the relevant arts to be attributable to measurement error, differences in measurement and/or manufacturing equipment calibration, human error in reading and/or setting measurements, adjustments made to optimize performance and/or structural parameters in view of differences in measurements associated with other components, particular implementation scenarios, imprecise adjustment and/or manipulation of objects by a person or machine, and/or the like.

1. A method comprising applying an effective amount of a composition to a honey bee (Apis melliferia) colony, the composition comprising one or more strains of Parasaccharibacter apium (P. apium), a supernatant of the one or more strains of P. apium, or a combination of the one or more strains of P. apium and the supernatant of the one or more strains of P. apium.

2. The method of claim 1, wherein the one or more strains of P. apium comprises strain SME1.

3. The method of claim 1, wherein the one or more strains of P. apium comprises one or more antibiotic resistant strains.

4. The method of claim 3, wherein at least one of the one or more antibiotic resistant strains is resistant to kanamycin.

5. The method of claim 3, wherein at least one of the one or more antibiotic resistant strains comprises an exogenous antibiotic resistance gene or a functional fragment thereof.

6. The method of claim 3, wherein at least one of the one or more antibiotic resistant strains comprises an exogenous kanamycin resistance gene or a functional fragment thereof.

7. The method of claim 6, wherein the exogenous kanamycin resistance gene or functional fragment thereof comprises a mini-Tn5 KanR cassette.

8. The method of claim 7, wherein the at least one of the one or more antibiotic resistant strains comprising the mini-Tn5 KanR cassette is an SME1 mutant.

9. The method according to claim 1, wherein applying the effective amount of the composition to a honey bee colony comprises at least one of: feeding the composition to honey bees of the honey bee colony; depositing the composition on a frame of the honey bee colony's hive, and depositing the composition on at least one surface or element within the honey bee colony's hive.

10. The method according to claim 1, wherein the composition is aqueous.

11. A composition comprising one or more strains of Parasaccharibacter apium (P. apium), a supernatant of the one or more strains of P. apium, or a combination of the one or more strains of P. apium and the supernatant of the one or more strains of P. apium.

12. The composition of claim 11, wherein the one or more strains of P. apium comprises strain SME1.

13. The composition of claim 11, wherein the one or more strains of P. apium comprises one or more antibiotic resistant strains.

14. The composition of claim 13, wherein at least one of the one or more antibiotic resistant strains is resistant to kanamycin.

15. The composition of claim 13, wherein at least one of the one or more antibiotic resistant strains comprises an exogenous antibiotic resistance transposon, gene, or a functional fragment thereof.

16. The composition of claim 13, wherein at least one of the one or more antibiotic resistant strains comprises an exogenous kanamycin resistance transposon, gene or a functional fragment thereof.

17. The composition of claim 16, wherein the exogenous kanamycin resistance gene or functional fragment thereof comprises a mini-Tn5 KanR cassette.

18. The composition of claim 17, wherein the at least one of the one or more antibiotic resistant strains comprising the mini-Tn5 KanR cassette is an SME1 mutant.

19. The composition according to claim 1, wherein the composition is aqueous.

20. The composition according claim 1, wherein the composition comprises a liquid carrier or a gel-based carrier.

21. The composition according to claim 1, wherein the composition comprises at least one carbon source.
